(19) United States
(12) Patent Application Publication
(10) Pub. No.: US 2008/0118560 A1
(43) Pub. Date: May 22, 2008

(54) Novel modified release formulation

(76) Inventor: Anne Juppo, Molndal (SE)

Correspondence Address: WHITE & CASE LLP;PATENT DEPARTMENT, 1155 AVENUE OF THE AMERICAS, NEW YORK, NY, 10036, US

(21) Appl. No.: 11/748,366

(22) Filed: May 14, 2007

Related U.S. Application Data
(63) Continuation of application No. 10/467,723, filed on Aug. 11, 2003, now abandoned.

(30) Foreign Application Priority Data
Feb. 13, 2001 (SE) 0100477-9
Feb. 13, 2001 (SE) 0100478.7

(51) Int. Cl. A61K 9/20; A61K 9/00; A61K 31/437; C07D 471/04; A61P 1/04

(52) U.S. Cl. 424/465; 424/484; 514/300; 546/121

(57) ABSTRACT

The present invention is directed to a multiparticulate, modified release solid dispersion formulation, comprising a drug substance having a pH-dependent solubility, said drug substance being a compound of the formula I, or a pharmaceutically acceptable salt thereof; a hydrophobic matrix former which is a water-insoluble, non-swelling amphiphilic lipid; and a hydrophilic matrix former which is a meltable, water-soluble excipient; wherein the weight ratio hydrophobic matrix former/hydrophilic matrix former is ≧1; and the particle size is less than 300 μm. Also a unit dosage of the same, as well as a process for the preparation thereof and the use of the formulation and unit dosage is claimed.

### FIELD OF THE INVENTION

The present invention is directed to a multiparticulate, modified release solid dispersion formulation, to a unit dosage of the same, as well as to a process for the preparation thereof. The invention also concerns the use of a multiparticulate, modified release solid dispersion formulation for the manufacture of a medicament for the treatment of gastric acid related diseases.

### BACKGROUND OF THE INVENTION

Solubility of a drug in the gastrointestinal fluids and its permeability through the cell membrane determines its oral bioavailability (Leuner and Dressman, Eur. J. Pharm. Biopharm 50, (2000) 47-60). For drugs with low aqueous solubility, the dissolution rate in the lumen is the rate-limiting step. Particle size reduction, solubilization, and salt formation are commonly used formulation methods to improve the dissolution rate. However, there are limitations to each of these techniques.

Many drugs do not only have low water solubility, but they might also have a narrow therapeutical index, which means that the drug levels in the blood have to be carefully controlled. This can be achieved by a controlled release formulation. They have other benefits compared to regular dosage forms; patient acceptability is usually better due to fewer doses per day, and the drug is usually more efficiently used so less active drug is needed.

Gel matrix tablets is a common drug form for modified release. The release rate is controlled either by erosion or by the diffusion of drug molecules in the swelled polymer matrix, which is the reason why drug solubility in the matrix material has great influence on the release rate. One disadvantage of matrix tablets is also that they cannot always be divided, whereas multiparticulate tablets can be divided.

Solid dispersions have been studied as a possibility to control the drug release rate (Aceves et al., Int. J. Pharm. 195, (2000) 45-53). Solid dispersion is a dispersion of one or more active ingredients in an inert carrier or matrix at solid state, prepared by the melting, (fusion), solvent or melting-solvent method (Chiou and Riegelman., J. Pharm. Sci. 60, (1971) 1281-1302). In J. Pharm. Sci. 58, (1969) 1505-1509, Chiou and Riegelman have classified the solid dispersions into following groups: Eutetic mixtures; solid solutions; glass solutions and glass suspensions; amorphous precipitations in crystalline carrier; and combinations of those above.

Melt processing (fusion method) was presented for the first time by Segikuchi, K. and Obi, N. in 1961, in Chem. Pharm. Bull. 9 (1961), 866-872 to prepare solid dispersions. In the melt method a physical mixture of the carrier and the drug is melted and then solidified. Cooling leads to supersaturation, but due to solidification the dispersed drug is trapped in to the carrier matrix. Melt method is often recommended, because no organic solvents are needed, so it is often less costly and better for the environment than the solvent method. However, it is not a suitable manufacturing method for thermolabile drugs. Thermal degradation, sublimation and polymorphic transformations may also occur during fusion (Goldberg et al, J. Pharm. Sci. 54, (1965) 1145-1148).

The principle of solid dispersions has been used in many pharmaceutical formulations, mostly in order to increase the bioavailability but in some cases for obtaining sustained release. Solid dispersions can be prepared of lipophilic matrix materials. The release rate is adjusted by varying the drug-excipient ratio. The amount of drug released increases with increased loading (Bodmeier et al, Drug. Dev. Ind. Pharm. 16 (9), (1990) 1505-1519).

Besides waxes and polar lipids, different polymers have been used to control drug release rate from solid dispersions. Ozeki et al. have shown that the release rate of phenacetin from a solid dispersion composed of poly(ethylene oxide)-carboxyvinylpolymer interpolymer complex can be controlled (Ozeki et al., J. Control. Release 58, (1999) 87-95).

U.S. Pat. No. 6,132,772 (corresponding to WO 96/23499) discloses an oral, extended release solid pharmaceutical composition comprising polyethylene glycol having a molecular weight of at least 1000, a drug having a solubility of less than 0.1% by weight in water at 20° C. and a hydrophilic gel-forming polymer having a mean molecular weight of at least 20 000.

U.S. Pat. No. 5,965,163 discloses a solid dosage form comprising a plurality of particles. The drug may according to this document be soluble or water insoluble.

U.S. Pat. No. 5,405,617 discloses the preparation of carrier matrices and spray congealed powders comprising an admixture of aliphatic or fatty acid esters and pharmaceutical actives which can be compressed into tablet and caplet dosage form.

U.S. Pat. No. 4,629,621 discloses a sustained release preparation of bioactive material having erodible characteristics.

Stearic acid (C-18) is the most commonly used of the fatty acids in pharmaceutical products. In oral pharmaceutical formulations, it is mainly used as a tablet lubricant in small concentrations and as a binder (The Handbook of Pharmaceutical Excipients, 3^{rd} Ed. AphA, (2000) 665). Stearic acid has also been used as a controlled release matrix excipient in spray congealing (Rodriguez et al., Int. J. Pharm 183, (1999) 133-143). The drug substances used by Rodriguez were theophylline having a water solubility at 25° C. of 8.3 mg/ml, and fenbufen having a water solubility at 25° C. of 0.11 mg/ml.

### OUTLINE OF THE INVENTION

The object of the present invention is to provide a pharmaceutical formulation of a drug substance having a pH-dependent solubility in water.

More particularly, the present invention is directed to a multiparticulate, modified release solid dispersion formulation, comprising

(i) a drug substance having a pH-dependent solubility, said drug substance being a compound of the formula I

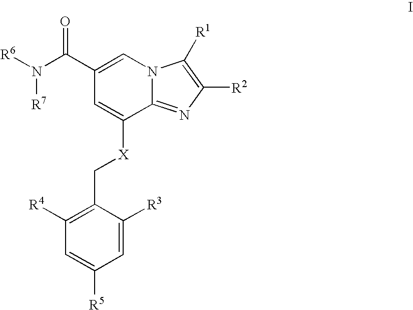

or a pharmaceutically acceptable salt thereof, wherein
R¹ is
   (b) CH₃, or
   (c) CH₂OH;
R² is
   (a) CH₃
   (b) CH₂CH₃
R³ is
   (a) H
   (b) C₁-C₆ alkyl,
   (c) hydroxylated C₁-C₆ alkyl
   (d) halogen
R⁴ is
(a) H,
(b) C₁-C₆ alkyl,
(c) hydroxylated C₁-C₆ alkyl, or
(d) halogen;
R⁵ is
   (a) H, or
   (b) halogen;
R⁶ and R⁷ are the same or different, selected from any one of
   (a) H,
   (b) C₁-C₆ alkyl;
   (c) hydroxylated C₁-C₆ alkyl
   (d) C₁-C₆ alkoxy-substituted C₁-C₆ alkyl
X is
   (a) NH, or
   (b) O;
      (ii) at least one hydrophobic matrix former which is a meltable, non-swelling amphiphilic lipid having a water-solubility below 1 mg/g; and
      (iii) at least one hydrophilic matrix former which is a meltable excipient having a water-solubility above 0.1 g/g;
      wherein
      the weight ratio hydrophobic matrix former/hydrophilic matrix former is ≧1; and
      the particle size is less than 300 μm.

The term “modified release” is herein defined as a formulation that releases less than 90% if its drug contents during the first three hours of the release.

The wording “at least one hydrophobic matrix former” as used herein, is defined such that one hydrophobic matrix former can be used alone, or in an alternative embodiment of the invention a mixture of hydrophobic matrix formers may be used.

The wording “at least one hydrophilic matrix former” as used herein, is defined such that one hydrophilic matrix former can be used alone, or in an alternative embodiment of the invention a mixture of hydrophilic matrix formers may be used.

The term “solid dispersion” is herein defined as a dispersion of the active compound of the formula I in an inert carrier or matrix at solid state. Solid dispersions are more particularly defined herein as eutetic mixtures, solid solutions, glass solutions or glass suspensions, amorphous precipitations in crystalline carrier or combinations thereof.

The solubility of the substances used in accordance with the present invention is pH-dependent. The wording “pH-dependent solubility” is in accordance with the present invention defined so as that the solubility of the drug substance in water is higher in lower pH, and lower in higher pH, more specifically at least 2 mg/ml at pH≦2 and lower than 1 mg/ml at pH≧4 at room temperature, i.e. at a temperature of about 23-25° C.

The wording “multiparticulate formulation” as used in accordance with the present invention is defined as a formulation comprising individual units of the drug substance, the hydrophobic matrix former and the hydrophilic matrix former, compressed into e.g. one single tablet.

The hydrophobic matrix formers are in accordance with the present invention water-insoluble, non-swelling fatty acids having a melting point above 50° C., more particularly a melting point up to 55° C. Examples of a specific fatty acid useful in accordance with the present invention is myristic acid.

The hydrophilic matrix formers are in accordance with the present invention meltable, water soluble excipients which are solid at room temperature, such as polyethylene oxides; polyethylene glycols; and polyethylene oxide and polypropylene oxide block-co-polymers, e.g. poloxamers. Specific examples of poloxamers useful in accordance with the present invention are poloxamer 188, also known under the trade name Pluronic F68®, and poloxamer 407, which is also known under the trade name Pluronic F127®. Pluronic F68® and Pluronic F127® are commercially available from BASF. Specific examples of polyethylene glycols useful in accordance with the present invention are PEG 4000, known under the trade name Macrogol 4000®, and PEG 6000, known under the trade name, Macrogol 6000®. Any poloxamer and PEG which are solid at room temperature may be used in accordance with the present invention. A comprehensive list of poloxamers and PEG's useful in accordance with the present invention can be found in Handbook of Pharmaceutical Excipients 3rd Ed., American Pharmaceutical Association and Pharmaceutical Press (2000), Washington, 665, which is hereby incorporated by reference, but which list however should not in any way be interpreted as exhaustive. Also other hydrophilic excipients which are miscible with the hydrophobic matrix formers as melts are useful in accordance with the present invention.

The weight ratio of hydrophobic matrix former/hydrophilic matrix former is ≧1, the excess amount of the hydrophobic matrix providing a sustained release effect.

In one embodiment of the invention, the total amount of the compound of the formula I is below about 40% by weight. In a further aspect of the invention the total amount of the compound of the formula I is 30-40% by weight, and in still a further embodiment of the invention the total amount of the compound of the formula I is 20-30% by weight.

The wording “unit dosage form” is herein defined as a composition where the amount of active compound of the formula I is administered as one single tablet, capsule or other suitable form in accordance with the present invention.

As used herein, the term “C₁-C₆ alkyl” denotes a straight or branched alkyl group having from 1 to 6 carbon atoms. Examples of said C₁-C₆ alkyl include methyl, ethyl, n-propyl, iso-propyl, n-butyl, iso-butyl, sec-butyl, t-butyl and straight- and branched-chain pentyl and hexyl.

The term “halogen” includes fluoro, chloro, bromo and iodo.

As the drug substance used in accordance with the present invention are both the pure enantiomers, racemic mixtures and unequal mixtures of two enantiomers. It should be understood that all the diastereomeric forms possible (pure enantiomers, racemic mixtures and unequal mixtures of two enantiomers) are within the scope of the present invention as the active drug substance, as well as derivatives of the compounds of the Formula I which have the biological function of the compounds of the Formula I, such as prodrugs.

It will also be appreciated by those skilled in the art, although derivatives of compounds of formula I may not possess pharmacological activity as such, they may be administered orally and thereafter metabolised in the body to form compounds of the invention which are pharmacologically active. Such derivatives may therefore be described as “prodrugs”. Prodrugs of compounds of formula I are also within the scope of the invention. Depending on the process conditions the end products of the Formula I are obtained either in neutral or salt form. Both the free base and the salts of these end products are within the scope of the invention.

In one aspect of the invention, the active drug substance is a compound of the formula I wherein R¹ is CH₃ or CH₂OH; R² is CH₃ or CH₂CH₃; R³ is CH₃ or CH₂CH₃; R⁴ is CH₃ or CH₂CH₃; R⁵ is H, Br, Cl, or F.

In a further aspect of the present invention, the active drug substance of the formula I is a compound selected from any one of
2,3-dimethyl-8-(2-ethyl-6-methylbenzylamino)-N-propyl-imidazo[1,2-a]pyridine-6-carboxamide;
8-(2-ethyl-6-methylbenzylamino)-3-hydroxymethyl-2-methylimidazo[1,2-a]pyridine-6-carboxamide;
2,3-dimethyl-8-(2,6-dimethylbenzylamino)-N-hydroxyethyl-imidazo[1,2-a]pyridine-6-carboxamide;
2,3-dimethyl-8-(2-ethyl-6-methylbenzylamino)-imidazo[1,2-a]pyridine-6-carboxamide;
8-(2-ethyl-6-methylbenzylamino)-N,2,3-trimethylimidazo[1,2-a]pyridine-6-carboxamide;
8-(2-ethyl-6-methylbenzylamino)-N,N,2,3-tetramethylimidazo[1,2-a]pyridine-6-carboxamide;
2,3-dimethyl-8-(2,6-dimethylbenzyl-amino)-imidazo[1,2-a]pyridine-6-carboxamide, N-[2-(dimethylamine)-2-oxoethyl]-8-(2-ethyl-6-methylbenzylamino)-N,2,3-trimethylimidazo[1,2-a]pyridine-6-carboxamide;
2,3-dimethyl-8-(2-ethyl-4-fluoro-6-methylbenzylamino)-imidazo[1,2-a]pyridine-6-carboxamide mesylate;
2,3-dimethyl-8-(2-methylbenzylamino)-imidazo[1,2-a]pyridine-6-carboxamide;
2,3-dimethyl-8-(2,6-dimethyl-4-fluoro-benzylamino)-imidazo[1,2-a]pyridine-6-carboxamide mesylate;
2,3-dimethyl-8-(2-methyl-6-isopropylbenzylamino)-imidazo[1,2-a]pyridine-6-carboxamide mesylate;
2,3-dimethyl-8-(2,6-diethyl-benzylamino)-imidazo[1,2-a]pyridine-6-carboxamide;
2,3-dimethyl-8-(2-ethylbenzylamino)-imidazo[1,2-a]pyridine-6-carboxamide;
2,3 dimethyl-8-(2-ethyl-6-methyl-benzylamino)-N-hydroxyethyl-imidazo[1,2-a]pyridine-6-carboxamide;
N-(2,3-dihydroxypropyl)-2,3-dimethyl-g-(2-ethyl-6-methylbenzylamino)-[1,2-a]pyridine-6-carboxamide;
2,3 dimethyl-8-(2-ethyl-6-methyl-benzylamino)-N-(2-methoxyethyl)-imidazo[1,2-a]pyridine-6-carboxamide;
2-methyl-8-(2-ethyl-6-methylbenzylamino)-imidazo[1,2-a]pyridine-6-carboxamide;
2,3-dimethyl-8-(2-bromo-6-methylbenzylamino)-imidazo[1,2-a]pyridine-6-carboxamide;
2,3-dimethyl-8-(2-(2-hydroxyethyl)-6-methylbenzylamino)-imidazo[1,2-a]pyridine-6-carboxamide;
8-(2-ethyl-6-methylbenzylamino)-N,N-bis(2-hydroxyethyl)-2,3-dimethylimidazo[1,2-a]pyridine-6-carboxamide;
8-(2-ethyl-6-methylbenzylamino)-N-(2-hydroxyethyl)-N,2,3-trimethylimidazo[1,2-a]pyridine-6-carboxamide; and
2,3-dimethyl-8-(2-ethyl-6-methylbenzyloxy)-imidazo[1,2-a]pyridine-6-carboxamide; or a pharmaceutically acceptable salt thereof.

In still a further aspect of the present invention, the active drug substance of the formula I is a compound selected from any one of
8-(2-ethyl-6-methylbenzylamino)-3-hydroxymethyl-2-methylimidazo[1,2-a]pyridine-6-carboxamide;
2,3-dimethyl-8-(2,6-dimethylbenzylamino)-N-hydroxyethyl-imidazo[1,2-a]pyridine-6-carboxamide;
2,3-dimethyl-8-(2-ethyl-6-methylbenzylamino)-imidazo[1,2-a]pyridine-6-carboxamide;
8-(2-ethyl-6-methylbenzylamino)-N,2,3-trimethylimidazo[1,2-a]pyridine-6-carboxamide;
2,3-dimethyl-8-(2,6-dimethylbenzylamino)-imidazo[1,2-a]pyridine-6-carboxamide;
2,3-dimethyl-8-(2-ethyl-4-fluoro-6-methylbenzylamino)-imidazo[1,2-a]pyridine-6-carboxamide;
2,3-dimethyl-8-(2,6-dimethyl-4-fluoro-benzylamino)-imidazo[1,2-a]pyridine-6-carboxamide;
2,3-dimethyl-8-(2,6-diethylbenzylamino)-imidazo[1,2-a]pyridine-6-carboxamide;
2,3-dimethyl-8-(2-ethyl-6-methylbenzylamino)-N-hydroxyethyl-imidazo[1,2-a]pyridine-6-carboxamide; and
2,3-dimethyl-8-(2-ethyl-6-methylbenzylamino)-N-(2-methoxyethyl)-imidazo[1,2-a]pyridine-6-carboxamide; or a pharmaceutically acceptable salt thereof.

Compounds of the formula I above and their preparation, are described in the patent application PCT/SE99/00663 (WO 99/55706), which is hereby incorporated in full as reference.

The pharmaceutical formulation according to the present invention is useful particular for the inhibition of gastric acid secretion. Thus, one aspect of the present invention is the use of a multiparticulate, modified release formulation as claimed and described herein, for the manufacture of a medicament for the inhibition of gastric acid secretion.

Another aspect of the present invention, is a method for the inhibition of gastric acid secretion, whereby a multiparticulate, modified release formulation as claimed and described herein, is administered to a patient in need of such gastric acid secretion inhibition.

The typical daily dose of the active substance according to formula I above varies within a wide range and will depend on various factors such as for example the individual requirement of each patient and the disease. In general, the formulation according to the invention is administered orally, and dosages will be in the range of from 5 to 1000 mg per day of active substance.

The multiparticulate, modified release formulation according to the present invention may be formulated into a unit dosage form, preferably as a tablet, which may also comprise standard excipients known to the skilled person in the art of tablet formulation. Examples of such excipients are fillers, binders, disintegrants and lubricants, but this list should however not be interpreted as being exhaustive.

The multiparticulate, modified release solid dispersion formulation according to the present invention provides the possibility of formulating drug substances of the formula I, said drug substances having a pH-dependent solubility in water. The novel formulation is particularly useful when formulated into a tablet, since it may also provide improved chances of dividing the tablet without disturbing the release rate of the active drug substance.

### Methods of Preparation

In spray congealing, or spray chilling as it is also called, the melted mass is atomized into droplets, which solidify quickly in cool air (Killeen, Pharm. Eng., July/August 1993, 56-64). The process differs from spray drying in that in spray drying the main action is evaporation of solvent caused by warm air, whereas in spray congealing it is a phase change from liquid to solid.

The spray congealing process used in accordance with the present invention comprises the following steps:

(i) melting the hydrophobic matrix former;
(ii) partially or totally dissolving, or emulsifying the compound of formula I into the melt;
(iii) dissolving the hydrophilic matrix former into the melt;
(iv) atomizing the melt into droplets;
(v) solidifying the droplets; and
(vi) collecting the particles.

The produced particles can then be further formulated into tablets or filled into capsules.

By the wording “partially or totally dissolving” in the method step (ii) above, it should be understood that a slight amount of the compound may remain undissolved in the melt. The atomization into droplets can be done with different-techniques, such as with a capillary nozzle, with a pneumatic nozzle, with an ultrasonic nozzle, with a hydraulic nozzle, with electrospraying, with rotary atomization, and preferably with a pneumatic nozzle using warm air as atomization gas.

The solidification of droplets can take place in liquid nitrogen, in or on carbondioxide ice or in air with a temperature lower than the melting point of the droplets. The particles may be collected into a vessel directly, or with a cylinder connected to a cyclone. The resulted particles are smaller than 300 μm, preferably spherical. The drug is in the particles present in the form of a solid dispersion.

Additives may be added into the melt prior to the atomization. Examples of such additives are surface active agents, excipients increasing viscosity, and buffering agents, but this list should however not in any way be interpreted as limiting the invention.

### DETAILED DESCRIPTION OF THE INVENTION

The invention will now be described in more detail by way of the following examples, which however should not be construed as limiting the invention in any way.

The following multiparticulate, modified release solid dispersion formulations were prepared.

| Example 1 | amount [g] |
|---|---|
| (i) 2,3-dimethyl-8-(2-ethyl-6- | 1 |
| methylbenzylamino)imidazo[1,2-a]- | |
| pyridine-6-carboxamide mesylate | |
| (ii) myristic acid | 4 |
| (iii) PEG 4000 | 2 |

### I. Preparation of the Multiparticulate, Modified Release Formulation

2,3-dimethyl-8-(2-ethyl-6-methylbenzylamino)imidazo[1,2-a]-pyridine-6-carboxamide mesylate (1 g) was dissolved in a melt of 4-g myristic acid at 90° C. The amount of 2 g polyethylene glycol 4000 (PEG 4000) was added into the melt. The melted mixture was kept at 90° C. and atomized with a pneumatic nozzle having an inner diameter of 1 mm and by using atomization air temperature of 400° C. and a pressure of 7 bar. The particles were collected into a vessel which was kept on carbondioxide ice (temperature −50° C.).

The resulted particles were spherical and smaller than 300 μm in size, as seen in Scanning Electron Micrograph (SEM).

### II. Tabletting

The amount of 3 g of particles prepared in step I above, were blended with 5.85 g microcrystalline cellulose and 0.016 g sodium stearyl fumarate in a Turbula mixer of the type 72C, Willy A. Bachofen AG Maschinenfabrik, Basle, Switzerland, for 10 minutes. This mixture was compressed by using an excentric tablet press Korsch EK-0 into 450 mg tablets using 11.3 mm flat punches with a maximum compression force of 5.0-5.6 kN. The breaking force of resulting tablets was measured by using a Schleuniger tablet hardness tester 4M, Dr. Schleuniger Productronic AG, Solothurn, Switzerland. The breaking force of resulting tablets was within the range 139-168 N.

The dissolution of tablets was tested with USP II paddle method in 900 ml 0.1 M HCl with 50 rpm. The amount dissolved in 3 hours was from 52-56%.

| Example 2 | amount [g] |
|---|---|
| (i) 2,3-dimethyl-8-(2-ethyl-6- | 1 |
| methylbenzylamino)imidazo[1,2-a]- | |
| pyridine-6-carboxamide mesylate | |
| (ii) myristic acid | 4 |
| (iii) PEG 4000 | 2 |

### I. Preparation of the Multiparticulate, Modified Release Formulation

2,3-dimethyl-8-(2-ethyl-6-methylbenzylamino)imidazo[1,2-a]-pyridine-6-carboxamide mesylate (1 g) was dissolved in a melt of 4 g myristic acid at 90° C. The amount of 2 g polyethylene glycol 4000 (PEG 4000) was added into the melt. The melted mixture was kept at 90° C. and atomized with a pneumatic nozzle having an inner diameter of 1 mm and by using atomization air temperature of 400° C. into a cylinder in room temperature. The cylinder was connected to a cyclone, which was used to collect the particles.

The resulted particles were spherical and smaller than 300 μm in size, as seen in Scanning Electron Micrograph (SEM).

1. A multiparticulate, modified release solid dispersion formulation, comprising
(i) a drug substance having a pH-dependent solubility, said drug substance being a compound of the formula I 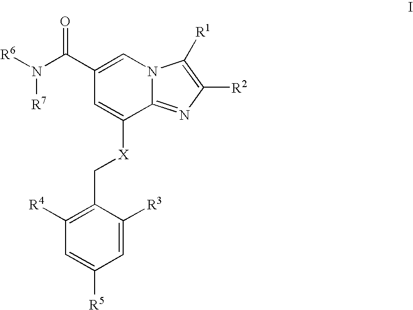
or a pharmaceutically acceptable salt thereof, wherein:
R¹ is
(a) H,
(b) CH₃, or
(c) CH₂OH;
R² is
(a) CH₃, or
(b) CH₂CH₃;
R³ is
(a) H,
(b) C₁-C₆ alkyl,
(c) hydroxylated C₁-C₆ alkyl, or
(d) halogen;
R⁴ is
(a) H,
(b) C₁-C₆ alkyl,
(c) hydroxylated C₁-C₆ alkyl, or
(d) halogen;
R⁵ is
(a) H, or
(b) halogen;
R⁶ and R⁷ are the same or different, selected from any one of
(a) H,
(b) C₁-C₆ alkyl,
(c) hydroxylated C₁-C₆ alkyl, and
(d) C₁-C₆ alkoxy-substituted C₁-C₆ alkyl; and
X is
(a) NH, or
(b) O;
(ii) at least one hydrophobic matrix former which is a meltable, non-swelling amphiphilic lipid having a water-solubility below 1 mg/g; and
(iii) at least one hydrophilic matrix former which is a meltable excipient having a water-solubility above 0.1 g/g;
wherein
the weight ratio of the hydrophobic matrix former to the hydrophilic matrix former is ≧1; and
the particle size is less than 300 μm.

2. The multiparticulate, modified release solid dispersion formulation according to claim 1, wherein the solubility of the drug substance in water is at least 2 mg/ml at pH≦2 and at room temperature.

3. The multiparticulate, modified release solid dispersion formulation according to claim 1, wherein the solubility of the drug substance in water is lower than 1 mg/ml at pH≧4 and at room temperature.

4. The multiparticulate, modified release solid dispersion formulation according to claim 1, wherein the hydrophobic matrix former is a water-insoluble, non-swelling fatty acid having a melting point above 50° C.

5. The multiparticulate, modified release solid dispersion formulation according to claim 1, wherein the hydrophobic matrix former is a water-insoluble, non-swelling fatty acid having a melting point of up to 55° C.

6. The multiparticulate, modified release solid dispersion formulation according to claim 1, wherein the hydrophobic matrix former or mixture thereof, comprises myristic acid.

7. The multiparticulate, modified release solid dispersion formulation according to claim 1, wherein the hydrophilic matrix former is selected from the group consisting of polyethylene oxides, polyethylene glycols, polyethylene oxides and polypropylene oxide block-co-polymers.

8. The multiparticulate, modified release solid dispersion formulation according to claim 7, wherein the hydrophilic matrix former is a poloxamer.

9. The multiparticulate, modified release solid dispersion formulation according to claim 1, wherein the hydrophilic matrix former is a polyethylene glycol.

10. The multiparticulate, modified release solid dispersion formulation according to claim 7, wherein the hydrophilic matrix former or mixture thereof, is selected from PEG 4000 and PEG 6000.

11. The multiparticulate, modified release solid dispersion formulation according to claim 1, wherein R¹ is CH₃ or CH₂OH; R², R³ and R⁴ independently are CH₃ or CH₂CH₃; and R⁵ is H, Br, Cl, or F.

12. The multiparticulate, modified release solid dispersion formulation according to claim 1, wherein the compound of formula I is selected from the group consisting of:
2,3-dimethyl-8-(2-ethyl-6-methylbenzylamino)-N-propyl-imidazo[1,2-a]pyridine-6-carboxamide;
8-(2-ethyl-6-methylbenzylamino)-3-hydroxymethyl-2-methylimidazo[1,2-a]pyridine-6-carboxamide;
2,3-dimethyl-8-(2,6-dimethylbenzylamino)-N-hydroxyethyl-imidazo[1,2-a]pyridine-6-carboxamide;
2,3-dimethyl-8-(2-ethyl-6-methylbenzylamino)-imidazo[1,2-a]pyridine-6-carboxamide;
8-(2-ethyl-6-methylbenzylamino)-N,2,3-trimethylimidazo[1,2-a]pyridine-6-carboxamide;
8-(2-ethyl-6-methylbenzylamino)-N,N,2,3-tetramethylimidazo[1,2-a]pyridine-6-carboxamide;
2,3-dimethyl-8-(2,6-dimethylbenzyl-amino)-imidazo[1,2-a]pyridine-6-carboxamide;
N-[2-(dimethylamine)-2-oxoethyl]-8-(2-ethyl-6-methylbenzylamino)-N,2,3-trimethylimidazo[1,2-a]pyridine-6-carboxamide;
2,3-dimethyl-8-(2-ethyl-4-fluoro-6-methylbenzylamino)-imidazo[1,2-a]pyridine-6-carboxamide;
2,3-dimethyl-8-(2-methylbenzylamino)-imidazo[1,2-a]pyridine-6-carboxamide;
2,3-dimethyl-8-(2,6-dimethyl-4-fluoro-benzylamino)-imidazo[1,2-a]pyridine-6-carboxamide;
2,3-dimethyl-8-(2-methyl-6-isopropylbenzylamino)-imidazo[1,2-a]pyridine-6-carboxamide;
2,3-dimethyl-8-(2,6-diethyl-benzylamino)-imidazo[1,2-a]pyridine-6-carboxamide;
2,3-dimethyl-8-(2-ethylbenzylamino)-imidazo[1,2-a]pyridine-6-carboxamide;
2,3 dimethyl-8-(2-ethyl-6-methyl-benzylamino)-N-hydroxyethyl-imidazo[1,2-a]pyridine-6-carboxamide;
N-(2,3-dihydroxypropyl)-2,3 dimethyl-8-(2-ethyl-6-methylbenzylamino)-[1,2-a]pyridine-6-carboxamide;
2,3 dimethyl-8-(2-ethyl-6-methyl-benzylamino)-N-(2-methoxyethyl)-imidazo[1,2-a]pyridine-6-carboxamide;
2-methyl-8-(2-ethyl-6-methylbenzylamino)-imidazo[1,2-a]pyridine-6-carboxamide;
2,3-dimethyl-8-(2-bromo-6-methylbenzylamino)-imidazo[1,2-a]pyridine-6-carboxamide;
2,3-dimethyl-8-(2-(2-hydroxyethyl)-6-methylbenzylamino)-imidazo[1,2-a]pyridine-6-carboxamide;
8-(2-ethyl-6-methylbenzylamino)-N,N-bis(2-hydroxyethyl)-2,3-dimethylimidazo[1,2-a]-pyridine-6-carboxamide;
8-(2-ethyl-6-methylbenzylamino)-N-(2-hydroxyethyl)-N,2,3-trimethylimidazo-[1,2-a]pyridine-6-carboxamide; and
2,3-dimethyl-8-(2-ethyl-6-methylbenzyloxy)-imidazo[1,2-a]pyridine-6-carboxamide;
or a pharmaceutically acceptable salt thereof.

13. The multiparticulate, modified release formulation according to claim 12, wherein the compound is selected from the group consisting of:
8-(2-ethyl-6-methylbenzylamino)-3-hydroxymethyl-2-methylimidazo[1,2-a]pyridine-6-carboxamide;
2,3-dimethyl-8-(2,6-dimethylbenzylamino)-N-hydroxyethyl-imidazo[1,2-a]pyridine-6-carboxamide;
2,3-dimethyl-8-(2-ethyl-6-methylbenzylamino)-imidazo[1,2-a]pyridine-6-carboxamide;
8-(2-ethyl-6-methylbenzylamino)-N,2,3-trimethylimidazo[1,2-a]pyridine-6-carboxamide;
2,3-dimethyl-8-(2,6-dimethylbenzylamino)-imidazo[1,2-a]pyridine-6-carboxamide;
2,3-dimethyl-8-(2-ethyl-4-fluoro-6-methylbenzylamino)-imidazo[1,2-a]pyridine-6-carboxamide;
2,3-dimethyl-8-(2,6-dimethyl-4-fluoro-benzylamino)-imidazo[1,2-a]pyridine-6-carboxamide;
2,3-dimethyl-8-(2,6-diethylbenzylamino)-imidazo[1,2-a]pyridine-6-carboxamide;
2,3 dimethyl-8-(2-ethyl-6-methylbenzylamino)-N-hydroxyethyl-imidazo[1,2-a]pyridine-6-carboxamide; and
2,3 dimethyl-8-(2-ethyl-6-methylbenzylamino)-N-(2-methoxyethyl)-imidazo[1,2-a]pyridine-6-carboxamide;
or a pharmaceutically acceptable salt thereof.

14. The multiparticulate, modified release solid dispersion formulation according to claim 1, wherein the compound of formula I is in the form of a hydrochloride or mesylate salt.

15. The multiparticulate, modified release solid dispersion formulation according to claim 1, wherein the total amount of the drug substance of formula I of claim 1, is below about 40% by weight of the formulation.

16. A unit dosage form comprising a multiparticulate, modified release solid dispersion formulation according to any one of claims 1-15 and 23-26.

17. The unit dosage form according to claim 16, wherein the unit dosage form is a tablet, and optionally further comprises one or more pharmaceutically acceptable excipients.

18. The unit dosage form according to claim 17, wherein the excipients are microcrystalline cellulose and sodium stearyl fumarate.

19. A process for the preparation of a multiparticulate, modified release formulation according to any one of claims 1-15 and 23-26, comprising the step of spray congealing the formulation.

20. The process according to claim 19, whereby the spray congealing comprises the steps of:
(i) melting the hydrophobic matrix former;
(ii) partially or totally dissolving, or emulsifying, the compound of formula I into the melt;
(iii) dissolving the hydrophilic matrix former into the melt;
(iv) atomizing the melt into droplets;
(v) solidifying the droplets; and
(vi) collecting the particles.

21. (canceled)

22. A method for the inhibition of gastric acid secretion, comprising administering an effective amount of a multiparticulate, modified release solid dispersion formulation according to any one of claims 1-15 and 23-26 to a patient in need of such gastric acid secretion inhibition.

23. The multiparticulate, modified release solid dispersion formulation according to claim 1, wherein the hydrophobic matrix former is a mixture of hydrophobic matrix formers.

24. The multiparticulate, modified release solid dispersion formulation according to claim 1, wherein the hydrophilic matrix former is a mixture of hydrophilic matrix formers.

25. The multiparticulate, modified release solid dispersion formulation according to claim 12, wherein the compound is in the form of a hydrochloride or mesylate salt.

26. The multiparticulate, modified release solid dispersion formulation according to claim 13, wherein the compound is in the form of a hydrochloride or mesylate salt.
